(19) United States
(12) Patent Application Publication
(10) Pub. No.: US 2025/0308282 A1
(43) Pub. Date: Oct. 2, 2025

(54) HEART-RATE DETECTING METHOD IMPLEMENTED BY REMOTE PHOTOPLETHYSMOGRAPHY

(71) Applicant: GUANGZHOU LUXVISIONS INNOVATION TECHNOLOGY LIMITED, Guangzhou City (CN)

(72) Inventor: Chien-Hung TUNG, Guangzhou City (CN)

(21) Appl. No.: 18/913,250

(22) Filed: Oct. 11, 2024

(30) Foreign Application Priority Data
Mar. 28, 2024 (CN) 2024103681408

(51) Int. Cl. G06V 40/10; G06V 10/30; G06V 10/32; G06V 10/56; G06V 40/16; H04N 23/71; H04N 23/73

(52) U.S. Cl. CPC G06V 40/15; G06V 10/30; G06V 10/32; G06V 10/56; G06V 40/168; H04N 23/71; H04N 23/73

(57) ABSTRACT

A heart-rate detecting method implemented by remote photoplethysmography (rPPG) includes: continuously capturing input frames which include a facial image; computing multiple feature points in the facial image to obtain a skin-color average value and store the skin-color average value to a first queue; executing POS algorithm and CHROM algorithm based on the skin-color average values of the multiple input frames stored in the first queue to respectively generate a first rPPG wave signal and a second rPPG wave signal; performing a reverse-combining process to the first rPPG wave signal and the second rPPG wave signal to generate a combined wave signal; performing a Fast Fourier Transform process to the combined wave signal to generate a combined spectrum; and analyzing the combined spectrum to extract a heart-rate.

### BACKGROUND OF THE DISCLOSURE

### Technical Field

The disclosure generally relates to heart-rate detection, particularly to a heart-rate detecting method implemented by a contactless approach.

### Description of Related Art

Currently, various types of non-contact heart rate detectors are available on the market that can measure the heart rate of a user without physically contacting the user's body.

For example, some detectors use the Robust Pulse Rate from the Chrominance-based rPPG (CHROM) algorithm. The CHROM algorithm obtains the facial image of the user by a non-contact method and performs an image analysis on the facial image to obtain the ratio of RGB color changes to detect changes in blood volume in the blood vessels under the user's skin, and then estimates the user's pulse by the frequency of these changes.

Another example is some detectors that use the Plane-Orthogonal-to-Skin (POS) algorithm. The POS algorithm is derived from the CHROM algorithm whereas the difference is that the POS algorithm detects changes in different color dimension projection matrices.

However, the CHROM algorithm and the POS algorithm are respectively suitable for processing different skin colors and image brightness levels. Therefore, current detectors on the market that use only a single algorithm are not suitable for detecting the heart rate of all users because of different users, different usage environments, and different image-capturing units. As a result, the current detectors lead to inaccurate detection results in the case of detecting all users.

### SUMMARY OF THE INVENTION

The disclosure provides a heart-rate detecting method implemented by a remote Photoplethysmography (rPPG). By simultaneously referencing the rPPG signals generated by the CHROM algorithm and the POS algorithm, the accuracy of heart rate detection is improved.

In one embodiment, a heart-rate detecting method implemented by a remote Photoplethysmography (rPPG) performed by an electronic device including at least one image-capturing unit and a processor and including the following steps: continuously capturing input frames based on continuous time series by the image-capturing unit, where the input frames include a facial image; computing, by the processor, multiple feature points of the facial image to obtain a skin-color average and storing the skin-color average and a timestamp corresponding to the skin-color average to a first queue; performing, by the processor, a Plane-Orthogonal-to-Skin (POS) algorithm and a Robust Pulse Rate from Chrominance-based rPPG (CHROM) algorithm based on the skin-color average of the multiple input frames in the first queue to respectively generate a first rPPG wave signal and a second rPPG wave signal; performing, by the processor, a reverse merge process on the first rPPG wave signal and the second rPPG wave signal to generate a merged wave signal; performing, by the processor, a Fast Fourier Transform (FFT) on the merged wave signal to generate a combined spectrum; and performing, by the processor, a spectrum analysis process on the combined spectrum to retrieve a heart rate output.

Compared to the related art, the heart-rate detecting method of the disclosure accommodates a wider variety of skin-color samples and enhances the strength of the dominant frequency of the generated rPPG signals, so the accuracy of heart-rate detection results is improved.

### BRIEF DESCRIPTION OF THE DRAWINGS

FIG. 1 is a block diagram of an electronic device of one embodiment of the disclosure.

FIG. 2 is a flowchart illustrating a detecting method of one embodiment of the disclosure.

FIG. 3 is an illustration of a facial image of one embodiment.

FIG. 4 is a flowchart illustrating adjustments by an image-capturing unit of an embodiment of the disclosure.

FIG. 5 is a flowchart of creating a skin-color average of an embodiment of the disclosure.

FIG. 6A is a first schematic diagram of an rPPG wave signal of an embodiment of the disclosure.

FIG. 6B is a second schematic diagram of an rPPG wave signal of an embodiment of the disclosure.

FIG. 7 is a flowchart of a signal combination of an embodiment of the disclosure.

FIG. 8 is a schematic diagram of the first queue of an embodiment of the disclosure.

FIG. 9 is a spectrum diagram of an embodiment of the disclosure.

FIG. 10 is a flowchart of analyzing a spectrum of an embodiment of the disclosure.

### DETAILED DESCRIPTION

The disclosure provides a heart-rate detecting method implemented by a remote Photoplethysmography (rPPG) (referred to as “heart-rate detecting method” herein). The heart-rate detecting method may be implemented on any electronic device that captures a user's facial images. By analyzing the user's facial images, the heart-rate detecting method of the disclosure may directly detect a user's heart rate without contacting the user's body.

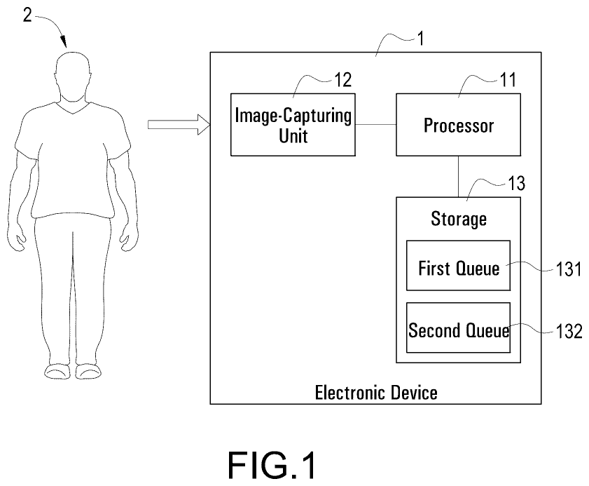

FIG. 1 is a block diagram of an electronic device of one embodiment of the disclosure. An electronic device 1 in FIG. 1 includes a processor 11, an image-capturing unit 12, and a storage 13. The processor 11 is electrically connected with the image-capturing unit 12 and the storage 13.

In one embodiment, the electronic device 1 may be a smart mobile phone, a tablet, a laptop, or a personal computer. The processor 11 may be a Digital Signal Processor (DSP), an Application Specific Integrated Circuit (ASIC), a Central Processing Unit (CPU), a System on Chip (SoC), a Field Programmable Gate Array (FPGA), or any combination of components above. The image-capturing unit 12 may be an RGB sensing unit or a camera. The storage 13 may be a Flash Memory, a Read Only Memory (ROM), a Hard Disk Drive (HDD), a Solid State Drive (SSD), or any combination of the components above. However, the listed components above are provided as embodiments and they are not limited herein.

In the heart-rate detecting method of the disclosure, the electronic device 1 controls the image-capturing unit 12 to continuously capture images of a user 2 whose heart rate is to be detected, performs at least two types of algorithms on the facial images at the same time to obtain the rPPG signals, and then computes the heart-rate of the user 2 according to the rPPG signals. Because the heart rate is computed by using the facial images, the detecting process is performed without contacting the body of the user 2. Furthermore, because at least two types of algorithms are implemented at the same time, a better detection result is provided no matter what skin color of the user 2 and the image brightness is.

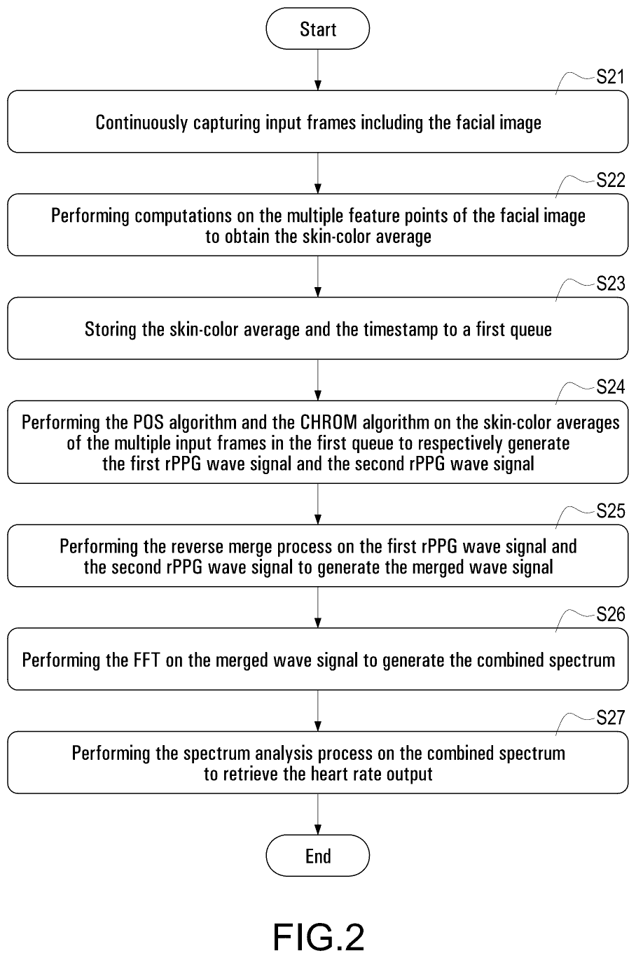

FIG. 2 is a flowchart illustrating a detecting method according to one embodiment of the disclosure. The detailed steps of the heart-rate detecting method are provided in FIG. 2 and may be performed by the electronic device 1 shown in FIG. 1, but are not limited herein.

As shown in FIG. 2, to perform the heart-rate detecting method, at first, the user 2 may use the electronic device (such as the electronic device 1 shown in FIG. 1) equipping the image-capturing unit 12 to take photographs and the image-capturing unit 12 continuously captures input frames (step S21). More particularly, the electronic device 1 shoots toward the user 2 to capture images to continuously obtain the input frames, and each input frame includes the image of the whole face of the user 2.

It should be noted that, in the heart-rate detecting method of the disclosure, the electronic device 1 continuously captures the facial image of the user 2 to continuously obtain multiple input frames, and the processor 11 of the electronic device 1 continuously performs steps below according to the multiple input frames. Accordingly, as long as the electronic device 1 continuously obtains the facial images of the user 2, the processor 11 continuously computes the heart rate of the user 2.

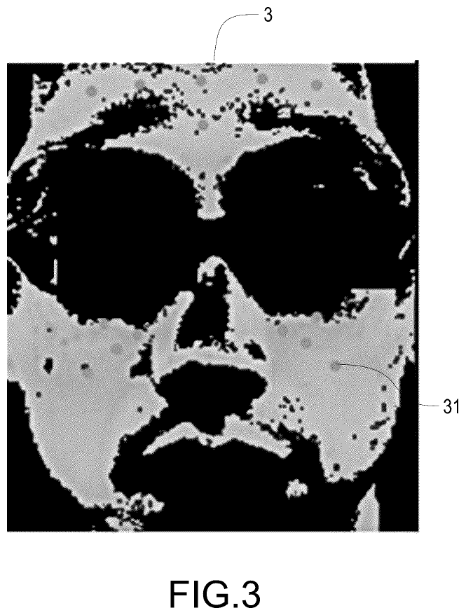

After step S21, the processor 11 performs an image analysis algorithm on the facial image (such as the facial image 3 shown in FIG. 3) of the input frame to identify multiple feature points (such as the multiple feature points 31 shown in FIG. 3) of the facial image, and performs computations on the multiple feature points to obtain a skin-color average of the facial image (step S22). Furthermore, the processor 11 stores the skin-color average and a timestamp corresponding to the skin-color average to a first queue 131 (step S23).

Reference is made to FIG. 3 incorporated with FIG. 1 and FIG. 2. FIG. 3 is an illustration of a facial image of one embodiment. In the disclosure, the processor 11 performs the image analysis algorithm to analyze the facial image 3 of the input frame to identify the multiple feature points 31 that are pre-set by the algorithm. The feature points are located on the flat region, such as the nasal wings (the left and/or right sides), cheeks (the left and/or right sides), and the forehead, which are not influenced when the head turns or moves. Although the user 2 turns his/her head when the image-capturing unit 12 captures images, the feature points are still acquired normally. The feature points are taken as the base data, so the accuracy of computing the skin-color average is enhanced.

In the disclosure, the electronic device 1 continuously captures images through the image-capturing unit 12, which the brightness of the input frame also affects the accuracy of the measured heart rate. More particularly, the heart-rate detecting method of the disclosure is suitable for some brightness ranges. Hence, the heart-rate detecting method of the disclosure may optionally adjust the image-capturing unit 12 that captures the images according to the computed skin-color average.

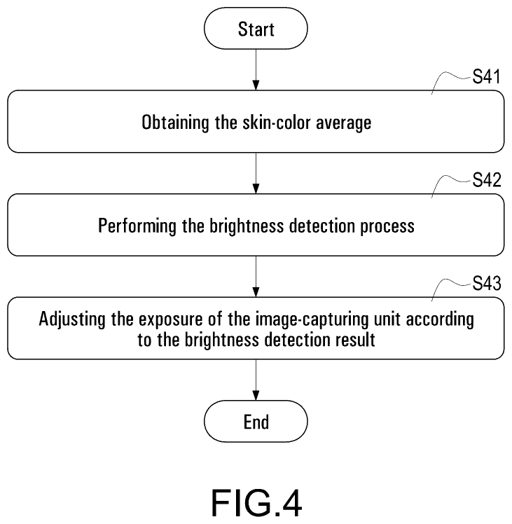

FIG. 4 is a flowchart illustrating adjustments by an image-capturing unit of an embodiment of the disclosure. In one embodiment, as shown in FIG. 4, the processor 11 obtains the skin-color average based on the multiple feature points on the facial image of the input frame (step S41) by performing step S22 in FIG. 2, then the processor 11 performs a brightness detection process on the skin-color average and generates a brightness detection result (step S42). The brightness detection process is an algorithm of determining whether the current skin-color average is enough to perform the image analysis or generate the rPPG signal that satisfies the quality requirement but is not limited herein. After step S42, the processor 11 sends the brightness detection result to the image-capturing unit 12 to directly adjust the exposure used by the image-capturing unit 12 capturing the next input frame (step S43). Specifically, the processor 11 adjusts one of physical parameters of the image-capturing unit 12 according to the brightness detection result to change the brightness of the facial image of the next input frame.

The brightness detection result includes an over-dark skin color, a moderate skin color, and an over-bright skin color. In step S43, the processor 11 controls the image-capturing unit 12 to increase the exposure when the brightness detection result shows that the skin color is over dark; the processor 11 controls the image-capturing unit 12 to decrease the exposure when the brightness detection result shows that the skin color is over bright; the processor 11 controls the image-capturing unit 12 to maintain the current exposure when the brightness detection result shows that the skin color is moderate.

Different from the related art, the disclosure does not perform the image process on the input frame, instead, directly adjusts the physical parameter used by the image-capturing unit 12 while capturing images, so the disclosure may more directly, more quickly, and more effectively optimize the obtained images.

As described above, the disclosure computes the skin-color average based on multiple default feature points (or called “multiple feature points”) on the facial image, however, a quantity of the default feature points of the algorithm is limited (such as 400 feature points), and it may be a problem of worse accurate while the skin-color average is computed by fewer feature points. Therefore, in one embodiment, the processor 11 may use skin-color pixels of the multiple default feature points and multiple sampling points around the multiple feature points (e.g., 8 sampling points around one feature point) to compute the skin-color average.

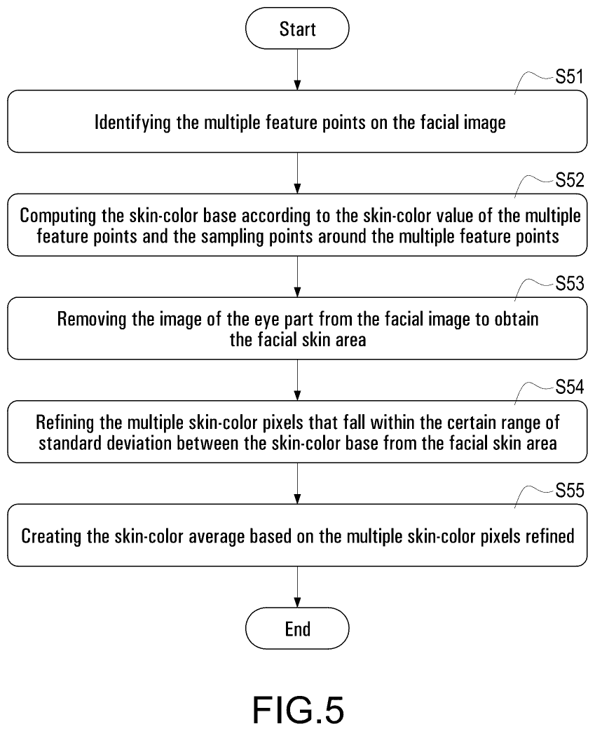

FIG. 5 is a flowchart of creating the skin-color average of an embodiment of the disclosure. FIG. 5 provides a more detailed illustration of step S22 in FIG. 2. As shown in FIG. 5, after obtaining an input frame, the processor 11 identifies the multiple feature points on the facial image by the algorithm (step S51), respectively captures skin-color values of the multiple sampling points around the multiple feature points, and computes a skin-color base according to the skin-color values of the multiple feature points and the sampling points (step S52).

After step S52, the processor 11 removes the image of the eye part from the facial image to obtain a facial skin area (step S53) and refines the multiple skin-color pixels that fall within a certain range of standard deviation between the skin-color base from the facial skin area (step S54). In the embodiment, the processor 11 creates the skin-color average based on the multiple skin-color pixels refined (step S55).

Specifically, the heart-rate detecting method of the disclosure first computes the skin-color base of multiple features on the facial image, and considers that the pixels with the skin-color base exceed the standard deviation may not be skin part of the user 2. Hence, the heart-rate detecting method of the disclosure screens out the pixels by the steps above. By only using the multiple skin-color pixels that are refined to create the skin-color average, it may enhance the accuracy of the skin-color average.

In the embodiment in FIG. 5, the processor 11 removes the image of the eye part from the facial image first, and then obtains the multiple skin-color pixels that satisfy the requirement from the processed facial image. However, in another embodiment, the processor 11 may directly obtain the multiple skin-color pixels from the facial image, and then remove the multiple skin-color pixels of the eye part from the multiple skin-color pixels. In other words, the steps S53 and S54 in FIG. 5 are not restricted to follow the execution order.

In one embodiment, the certain range of the standard deviation may be 1.5 times to 3 times (positive or negative) of the standard deviation. That is, the processor 11 refines the multiple skin-color pixels with the skin-color base are within 1.5 times to 3 times (positive or negative) of the standard deviation from the facial skin area and creates the skin-color average according to the skin-color pixels. However, the above description is only one of the embodiments of the disclosure but is not limited herein.

Referring to FIG. 2, after processor 11 receives and processes the multiple input frames, the multiple skin-color averages and the timestamps corresponding to the multiple skin-color averages are stored in the first queue 131. In the meantime, after step S23, the processor 11 performs the POS algorithm and the CHROM algorithm on the skin-color averages of the multiple input frames in the first queue 131 to respectively generate a first rPPG wave signal and a second rPPG wave signal (step S24). For ease of understanding, in the following statement, the result that the processor 11 computes the POS algorithm based on the skin-color averages of the multiple input frames in the first queue 131 is called “first rPPG wave signal”, and the result that the processor 11 performs the CHROM algorithm based on the skin-color averages of the multiple input frames in the first queue 131 is called “second rPPG wave signal”.

It should be noted that the POS algorithm and the CHROM algorithm applied in the disclosure are the POS algorithm and the CHROM algorithm whose outputs and superimposition window of the core algorithms are modified (described below).

After step S24, the processor 11 performs a reverse merge process on the first rPPG wave signal and the second rPPG wave signal to generate a merged wave signal (step S25). The heart-rate detecting method of the disclosure does not simply use the first rPPG wave signal generated by the POS algorithm to compute the heart rate of the user 2, nor does it simply use the second rPPG wave signal to compute the heart rate of the user 2. Instead, the processor 11 merges the first rPPG wave signal and the second rPPG wave signal first, and then computes the heart rate of the user 2 based on the merged wave signal. Therefore, the advantages of the POS algorithm and the CHROM algorithm are taken into account, so the result of the heart-rate detection is more accurate.

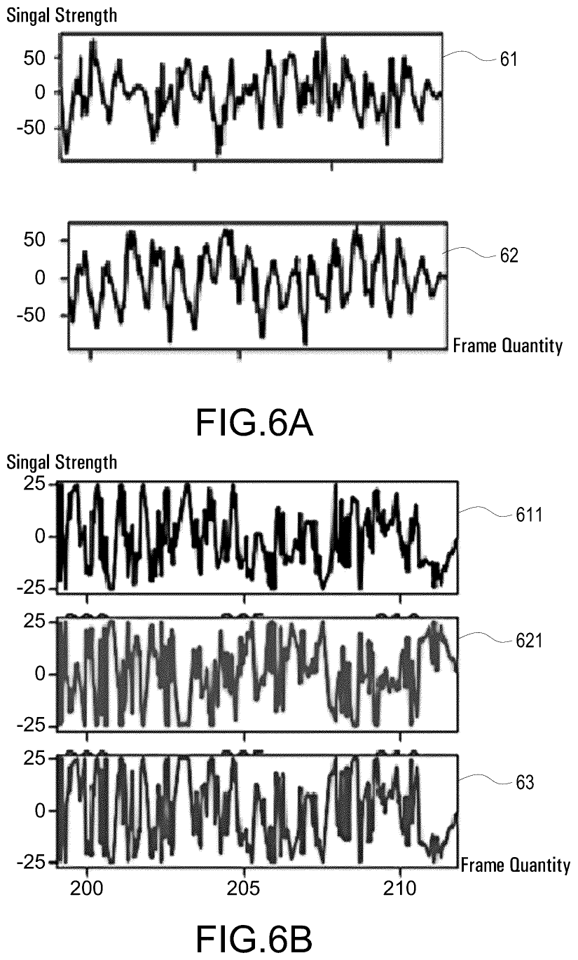

FIG. 6A is a first schematic diagram of an rPPG wave signal of an embodiment of the disclosure. The first wave signal in FIG. 6A is the first rPPG wave signal 61 generated by the processor 11 performing the POS algorithm, and the second wave signal is the second rPPG wave signal 62 generated by the processor 11 performing the CHROM algorithm.

By the experimental data shown in FIG. 6A, the POS algorithm and the CHROM algorithm applied by the disclosure have different vector values in the three-dimensional color space though, after outputting signals, the waves are similar and the frequencies are consistent while the directions are opposite. During the same time period (based on the frame numbers), it is not necessarily that the output signals of the POS algorithm and the CHROM algorithm are larger or smaller. To increase the strength of the effective signals that are applied in the rPPG, the disclosure takes the larger absolute value of frame nodes of the rPPG that are outputted by the two algorithms and merges signal strengths to generate the merged wave signal. By the reverse merge process on the first rPPG wave signal 61 and the second rPPG wave signal 62, the merged wave signal is suitable for variety types of skin-color samples, and the signal strength of the rPPG dominant frequency (i.e., the dominant frequency of the merged wave signal) is enhanced.

FIG. 6B is a second schematic diagram of an rPPG wave signal of an embodiment of the disclosure. In FIG. 6B, the first wave signal is the first rPPG wave signal 611 after an amplitude limiting process is performed on the first rPPG wave signal 61, the second wave signal is the second rPPG wave signal 621 after the amplitude limiting process is performed on the second rPPG wave signal 62, and the third wave signal is the merged wave signal 63 after the processor 11 performs the reverse merge process on the first rPPG wave signal 611 and the second rPPG wave signal 621. In one embodiment, in the heart-rate detecting method of the disclosure, before performing the reverse merge process, a bias calibration and the amplitude limiting process are performed on the first rPPG wave signal 61 and the second rPPG wave signal 62 first, and then the reverse merge process is performed on the processed first rPPG wave signal 611 and the second rPPG wave signal 621 to generate the merged wave signal 63 (detail description provided later).

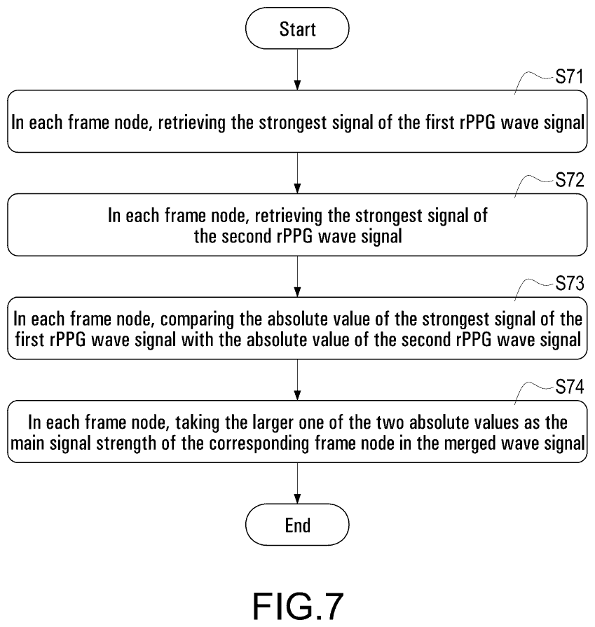

FIG. 7 is a flowchart of a signal combination of an embodiment of the disclosure. FIG. 7 is a more detailed description of step S25 in FIG. 2. For ease of description, the following statement takes the reverse merge process being performed on the first rPPG wave signal 61 and the second rPPG wave signal 62 to generate the merged wave signal 63 as an example, but the heart-rate detecting method of the disclosure may also generate the first rPPG wave signal 611 and the second rPPG wave signal 621 first and then perform the reverse merge process on the first rPPG wave signal 611 and the second rPPG wave signal 621 to generate the merged wave signal 63.

As shown in FIG. 7, after the POS algorithm and the CHROM algorithm are performed and the first rPPG wave signal 61 and the second rPPG wave signal 62 are obtained, in each frame node (i.e., each input frame), the processor 11 retrieves the strongest signal of the first rPPG wave signal 61 (step S71) and the strongest signal of the second rPPG wave signal 62 (step S72). Then, in each frame node, the processor 11 compares the absolute value of the strongest signal of the first rPPG wave signal 61 with the absolute value of the strongest signal of the second rPPG wave signal 62 (step S73) and takes the larger one of the two absolute values as the main signal strength of the corresponding frame node in the merged wave signal 63 (step S74). After comparing the absolute value of the strongest signal of the first rPPG wave signal 61 with the absolute value of the second rPPG wave signal 62 in each frame node, the merged wave signal 63 is generated, as shown in FIG. 6B.

As described above, the algorithm used in the disclosure is the modified POS algorithm and the modified CHROM algorithm. In one embodiment, the heart-rate detecting method of the disclosure may do further processing on the first rPPG wave signal 61 and the second rPPG wave signal 62 to generate the first rPPG wave signal 611 and the second rPPG wave signal 621 in FIG. 6B.

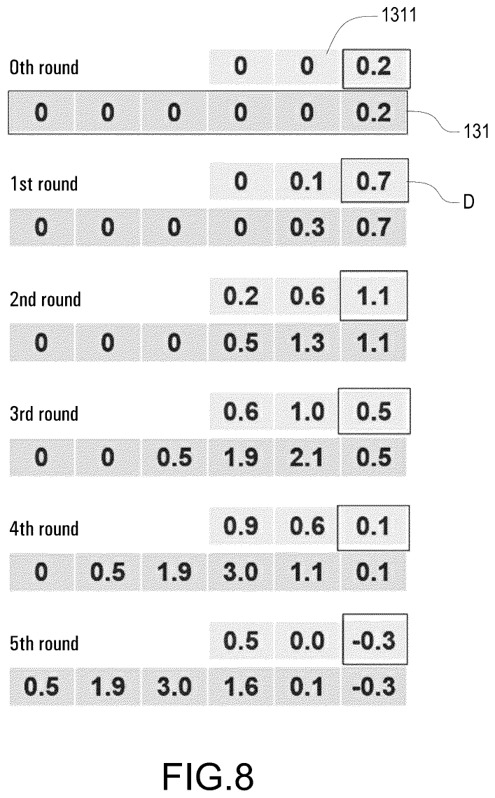

FIG. 8 is a schematic diagram of the first queue of an embodiment of the disclosure. FIG. 8 illustrates a specific embodiment of the first queue 131 of the disclosure. In one embodiment, the first queue 131 has a queue length M and stores M records of the skin-color averages based on the First In First Out (FIFO) principle. When the record quantity of the skin-color averages exceeds the queue length M, the processor 11 deletes the oldest record of the skin-color averages from the far left column of the first queue 131 and adds the latest record of the skin-color averages to the far right column of the first queue 131. In other words, the first queue 131 having queue length M may store the latest M records of the skin-color averages.

The first queue 131 has a superimposition window 1311. The superimposition window 1311 has a superimposition length N, and the superimposition length N is smaller than the queue length M. In one embodiment, the queue length M of the first queue 131 may be 250 frames, and the superimposition length N of the superimposition window 1311 may be 50 frames. An example that the queue length M is 6 and the superimposition length N is 3 is taken for ease of description, but the lengths shown in FIG. 8 are not limited herein.

The superimposition window 1311 is used to store the latest N records of the skin-color averages. Specifically, the POS algorithm and the CHROM algorithm of the disclosure generate the first rPPG wave signal 61 and the second rPPG wave signal 62 based on the skin-color averages in the first queue 131, and then the updated N records of the skin-color averages in the superimposition window 1311 are respectively superimposed on the latest N records of the skin-color averages in the first queue 131 after the latest skin-color average is respectively added to the first queue 131 and the superimposition window 1311.

As shown in FIG. 8, after the processor 11 computes and generates the first skin-color average (such as 0.2) (i.e., the 0th round), the processor 11 adds the latest skin-color average to the latest column (e.g., the far right column in FIG. 8) of the first queue 131 and the superimposition window 1311. In the meantime, before performing the POS algorithm and the CHROM algorithm based on the data of the first queue 131, the processor 11 superimposes the data (i.e., 0.2) of the first column in the superimposition window 1311 on the data (i.e., 0.2) of the first column in the first queue 131, superimposes the data (i.e., 0) of the second column in the superimposition window 1311 on the data (i.e., 0) of the second column in the first queue 131, superimposes the data (i.e., 0) of the third column in the superimposition window 1311 on the data (i.e., 0) of the third column in the first queue 131, and so on.

It should be noted that the queue length M takes the example of 250 frames and the superimposition length N takes the example of 50 frames, and because not all of the columns of the superimposition window 1311 are valid values before the electronic device 1 receives the first 50 frames, the computation of the data in the first queue 131 is not complete. Hence, in one embodiment, the processor 11 repeatedly performs step S21 to step S23 in the process of FIG. 2 until the frame quantity stored in the first queue 131 is greater than the superimposition length N (such as 50 frames) in the superimposition window 1311, and then proceeds to perform step S24 to compute the first rPPG wave signal 61 and the second rPPG wave signal 62.

On the other hand, because the processor 11 still superimposes the data of the superimposition window 1311 on the data of the corresponding column of the first queue 131 before the superimposition window 1311 is filled with data, the data of the first N frames (such as 50 frames) are not accurate. Hence, in another embodiment, the processor 11 repeatedly performs step S21 to step S23 in the process of FIG. 2 until the frame quantity is greater than the sum (such as 300 frames) of the superimposition length N of the superimposition window 1311 and the queue length M, and then proceed to perform step S24 to compute the first rPPG wave signal 61 and the second rPPG wave signal 62. In other words, the processor 11 does not compute the first rPPG wave signal 61 and the second rPPG wave signal 62 until the 50 oldest frames data in the first queue 131 are deleted.

Referring to the embodiment of FIG. 8, when the processor 11 computes and generates the second skin-color average (such as 0.7), the processor 11 shifts the data of the first queue 131 and the superimposition window 1311 to left one unit, and adds the latest skin-color average to the latest column of the first queue 131 and the superimposition window 1311. In the meantime, when performing the POS algorithm and the CHROM algorithm according to the data of the first queue 131, the processor 11 superimposes the data (i.e., 0.7) of the first column of the superimposition window 1311 on the data (i.e., 0.7) of the first column of the first queue 131, superimposes the data (i.e., 0.1) of the second column of the superimposition window 1311 on the data (i.e., 0.3) of the second column of the first queue 131, and superimposes the data (i.e., 0) of the third column of the superimposition window 1311 on the data (i.e., 0) of the third column of the first queue 131.

It should be noted that the step of adding the latest skin-color average to the superimposition window 1311 includes the processing procedure of alpha-tuning. Therefore, even though only one new record is stored in the superimposition window 1311, the computation results by the alpha-tuning based on the old data of the superimposition window 1311 in each round are different.

Because the signals are superimposed when the POS algorithm and the CHROM algorithm are performed, the bias will be amplified if the signal bias occurs. Hence, the signal bias is calibrated in each round before the signals are superimposed.

Specifically, before the POS algorithm and the CHROM algorithm are performed according to the data in the first queue 131, the processor 11 calibrates multiple latest frames of a first length D of the superimposition window 1311, where the calibration level of each frame in each round is 1/D of the average differences of the multiple frames, and the first length D is smaller than the superimposition length N. In one embodiment, the first length D may be one-fifth of the superimposition length N. Taking the superimposition length N as 50 frames as an example, the first length D is 10 frames. Before the heart-rate detecting method of the disclosure performs the superimposition, a small section bias calibration is performed on the data of one-fifth of the superimposition length N of the superimposition window 131. Taking the superimposition length N as 50 frames as one example, the length of the small section bias calibration is 10 frames. Because of the accumulation, the calibration quantity of each frame in each round is 1/10 of the average difference of 10 frames, such that the signals slightly tend to the average.

Referring to the embodiment of FIG. 8, after the processor 11 computes and generates the third skin-color average (such as 1.1), the processor 11 shifts the data of the first queue 131 and the superimposition window 1311 to left one unit, and adds the latest skin-color average to the latest column of the first queue 131 and the superimposition window 1311. When performing the superimposition, the processor 11 superimposes the data (i.e., 1.1) of the first column of the superimposition window 1311 on the data (i.e., 1.1) of the first column of the first queue 131, superimposes the data (i.e., 0.6) of the second column of the superimposition window 1311 on the data (i.e., 1.3) of the second column of the first queue 131, and superimposes the data (i.e., 0.2) of the third column of the superimposition window 1311 on the data (i.e., 0.5) of the third column of the first queue 131.

Similarly, after the processor 11 computes and generates the fourth skin-color average (such as 0.5), the processor 11 shifts the data of the first queue 131 and the superimposition window 1311 to left one unit, and adds the latest skin-color average to the latest column of the first queue 131 and the superimposition window 1311. When performing the superimposition, the processor 11 superimposes the data (i.e., 0.5) of the first column of the superimposition window 1311 on the data (i.e., 0.5) of the first column of the first queue 131, superimposes the data (i.e., 1.0) of the second column of the superimposition window 1311 on the data (i.e., 2.1) of the second column of the first queue 131, and superimposes the data (i.e., 0.6) of the third column of the superimposition window 1311 on the data (i.e., 1.9) of the third column of the first queue 131. It should be noted that the fourth column of the first queue 131 does not correspond to the superimposition window 1311, so the data (i.e., 0.5) of the fourth column of the first queue 131 is not calibrated.

After the processor 11 computes and generates the fifth skin-color average (such as 0.1) (i.e., the fourth round), the processor 11 shifts the data of the first queue 131 and the superimposition window 1311 to left one unit and adds the latest skin-color average to the latest column of the first queue 131 and the superimposition window 1311. When performing the superimposition, the processor 11 superimposes the data (i.e., 0.1) of the first column of the superimposition window 1311 on the data (i.e., 0.1) of the first column of the first queue 131, superimposes the data (i.e., 0.6) of the second column of the superimposition window 1311 on the data (i.e., 1.1) of the second column of the first queue 131, and superimposes the data (i.e., 0.9) of the third column of the superimposition window 1311 on the data (i.e., 3.0) of the third column of the first queue 131. After the processor 11 computes and generates the sixth skin-color average (such as −0.3) (i.e., the fifth round), the processor 11 shifts the data of the first queue 131 and the superimposition window 1311 to left one unit and adds the latest skin-color average to the latest column of the first queue 131 and the superimposition window 1311. When performing the superimposition, the processor 11 superimposes the data (i.e., −0.3) of the first column of the superimposition window 1311 on the data (i.e., −0.3) of the first column of the first queue 131, superimposes the data (i.e., 0.0) of the second column of the superimposition window 1311 on the data (i.e., 0.1) of the second column of the first queue 131, and superimposes the data (i.e., 0.5) of the third column of the superimposition window 1311 on the data (i.e., 1.6) of the third column of the first queue 131.

During the time the electronic device 1 continuously captures images and obtains the input frames, the processor 11 continuously performs the superimposition process on the data of the first queue 131 to update the data of the first queue 131. The heart-rate detecting method of the disclosure continuously updates the first queue 131 and performs the POS algorithm and the CHROM algorithm on the updated data of the first queue 131 to generate the first rPPG wave signal 61 and the second rPPG wave signal 62 based on the continuous time series.

It should be noted that before outputting the first rPPG wave signal 61 and the second rPPG wave signal 62, the processor 11 performs the amplitude limiting process on the first rPPG wave signal 61 and the second rPPG wave signal 62. By limiting the positive and negative signal strength of the first rPPG wave signal 61 and the second rPPG wave signal 62 to one-half of the original signal strength, the high-frequency noise and the low-frequency noise are filtered out. According to the embodiment of FIG. 6A and FIG. 6B, the upper/lower bound of the original signal strength of the first rPPG wave signal 61 and the second rPPG wave signal 62 is +50 to −50, and the signal strength of the first rPPG wave signal 611 and the second rPPG wave signal 621 is limited to +25 to −25 after the amplitude limiting process is performed.

Specifically, after the bias calibration is performed, the signals tend to the middle line, and then the amplitude limiting process is performed to limit the upper bound and the lower bound of the signal strength, so the little oscillations at the end of the signals are eliminated without affecting the signal accuracy, and the definite signals that are close to the square waves are outputted. In the disclosure, the first rPPG wave signal 611 and the second rPPG wave signal 621 are not outputted until the bias calibration and the amplitude limiting process are performed. Accordingly, the ideal heart rates/heartbeats are obtained based on the merged wave signal 63 generated by the first rPPG wave signal 611 and the second rPPG wave signal 621, so the processor 11 may directly compute the definite frequencies based on the merged wave signal 63 without performing any noise elimination process afterward.

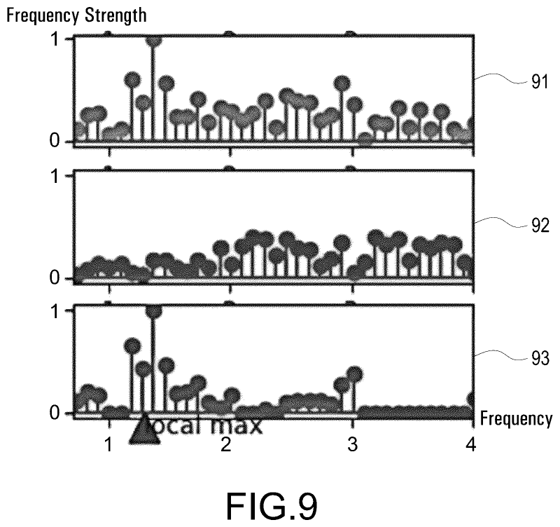

Referring to FIG. 2, after step S25, the processor 11 performs the Fast Fourier Transform (FFT) on the merged wave signal 63, transforming the merged wave signal 63 in the time domain into the combined spectrum in the frequency domain (step S26). Finally, the processor 11 performs the spectrum analysis process on the combined spectrum (such as the combined spectrum 91 of FIG. 9) to retrieve the heart rate output of the user 2 (step S27).

FIG. 9 is a spectrum diagram of an embodiment of the disclosure. In step S26 of FIG. 2, the processor 11 performs the FFT on the merged wave signal 63 to generate the frequency domain information, obtains the absolute value of the frequency domain information, and computes the normalization on the absolute value of the frequency domain information to obtain the combined spectrum 91. In the embodiment of FIG. 9, the X-axis of the spectrum is the frequency conversed based on the frame number and the time information of the first queue 131, taking 0 to 4 as the example; the Y-axis of the spectrum is the frequency strength after normalization, taking 0 to 1 as the example.

The data of 250 frames stored in the first queue 131 is taken as an example. After the merged wave signal 63 in the time domain is transformed into the combined spectrum 91 in the frequency domain, the processor 11 knows how long the current 250 frames occur and how many amplitudes it contains. Therefore, the processor 11 may compute how many frequencies in the spectrum to analyze the heart rate of the user 2. Specifically, after obtaining the definite spectrum, the processor 11 may compute the real frequency represented by the spectrum based on the frames being sampled with the timestamp corresponding to the first frame and the last frame. Furthermore, the processor 11 may retrieve the spectrum in a human heart rate range of about 0.66 Hz to 4 Hz (corresponding to 40 to 240 bpm), and then the actual current heart rate of the user 2 may be analyzed according to the strength distribution of each frequency.

It should be noted that the total quantity of the pixels in each input frame is different because the user 2 may move his/her head when the electronic device 1 continuously receives the images, and the difference in the total quantity of the pixels may affect the final retrieved spectrum. To optimize the spectrum basis for analyzing the heart rate, the heart-rate detecting method of the disclosure subtracts the difference in the total quantity of the pixels from the spectrum to optimize the spectrum, so the processor 11 may perform the spectrum analysis more accurately based on the optimized spectrum.

Specifically, in step S22 and step S23 in FIG. 2, the processor 11 analyzes the facial image to obtain the skin-color base and the multiple skin-color pixels that satisfy the standard deviation, creates the skin-color average based on the multiple skin-color pixels, and stores the skin-color average to the first queue 131. In one embodiment, the processor 11 stores the total quantity of the skin-color pixels of each input frame when analyzing the facial image, computes a skin-color-pixel variation between two adjacent frames in a time series (such as a previous frame and a next frame), and stores the skin-color-pixel variation in the second queue (e.g., the second queue 132 in FIG. 1).

In step S26 in FIG. 2, the processor 11 performs the FFT on the merged wave signal to generate the combined spectrum 91. Furthermore, the processor 11 performs the FFT on the skin-color-pixel variation of the multiple input frames in the second queue 132 to generate a skin-color-pixel variation spectrum 92.

In one embodiment, the processor 11 performs the spectrum analysis directly on the combined spectrum 91 to retrieve the heart rate output of the user 2. In another embodiment, the processor 11 subtracts the skin-color-pixel variation spectrum 92 from the combined spectrum 91 to generate a final spectrum 93, and then the processor 11 performs the spectrum analysis on the final spectrum 93 to obtain the heart rate output of the user 2. By directly subtracting skin-color-pixel variation spectrum 92 from the main spectrum (i.e., the combined spectrum 91) that is generated based on the rPPG signals, the clearer dominant frequency is obtained, so the processor 11 may retrieve the accurate heart rate easily.

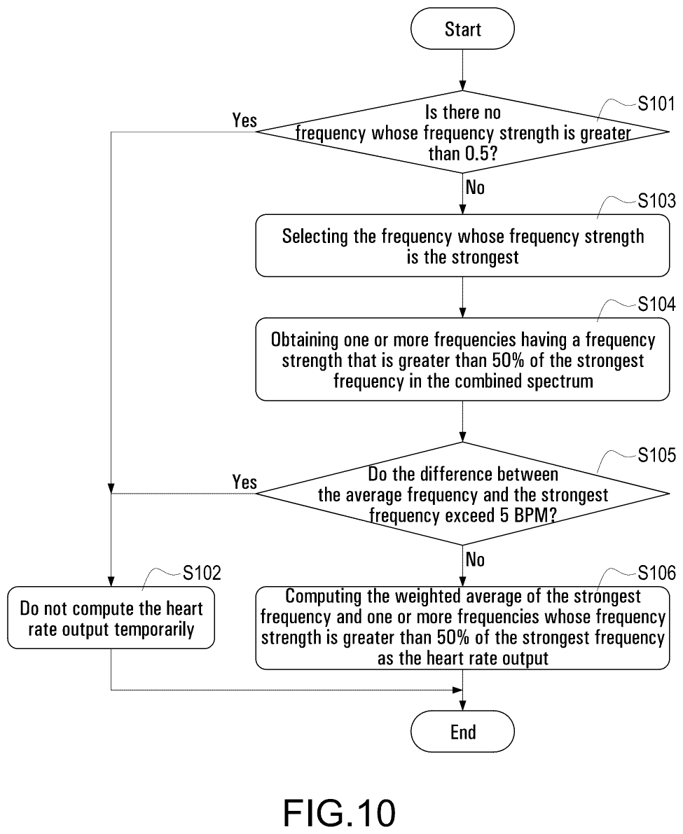

As illustrated in FIG. 9, the strongest frequency is centralized at frequencies around 1.2 Hz to 1.3 Hz. Accordingly, the heart-rate detecting method of the disclosure applies the following processes to retrieve the heart rate output of the user 2 from the final spectrum 93. FIG. 10 is a flowchart of analyzing a spectrum of an embodiment of the disclosure. As described above, the processor 11 performs the spectrum analysis on the combined spectrum 91 or the final spectrum 93 to retrieve the heart rate output of the user 2. For the sake of explanation, the following statement takes the processor 11 performing the spectrum analysis on the combined spectrum 91 as one example, but it is not limited herein.

In the embodiment in FIG. 10, first, the processor 11 determines whether the frequency whose frequency strength is greater than 0.5 is not included in the combined spectrum 91 (step S101). If there is no frequency whose frequency strength is greater than 0.5 included in the combined spectrum 91, the processor 11 temporarily does not compute the heart rate output (step S102) because it is difficult to compute the heart rate output without obvious dominant frequency.

If it is determined that the combined spectrum 91 includes at least one frequency whose frequency strength is greater than 0.5 (i.e., the determination is “No” in step S101), the processor 11 obtains the strongest frequency of the combined spectrum 91 (step S103). The combined spectrum 91 of FIG. 9 is taken as an example. Because the strongest frequency in the combined spectrum 91 is 1, in step S103, the processor 11 selects the frequency whose frequency strength is 1 as the strongest frequency. At the same time, the processor 11 obtains one or more frequencies having a frequency strength that is greater than 50% of the strongest frequency in the combined spectrum 91 (step S104). For example, if the strength of the strongest frequency is 0.8, in step S104, the processor 11 may only obtain one or more frequencies in the combined spectrum 91 whose frequency strength is greater than 0.4, while frequencies whose frequency strength is smaller than or equal to 0.4 are not used.

Next, the processor 11 computes the average frequency of the combined spectrum 91 based on the one or more frequencies obtained in step S104 and computes the difference between the average frequency and the strongest frequency. The difference is converted into the heart rate in Beats Per Minute (BPM), and it is determined whether the difference between the average frequency and the strongest frequency exceeds 5 BPM (step S105). If it is determined in step S105 that the difference between the average frequency and the strongest frequency exceeds 5 BPM, it indicates that the current signal is not stable (e.g., there are multiple dominant frequencies), so the processor 11 temporarily does not compute the heart rate output (step S102). Meanwhile, the electronic device 1 continuously captures images by the image-capturing unit 12, and the processor 11 continuously generates the first rPPG wave signal 61, the second rPPG wave signal 62, the first rPPG wave signal 611, the second rPPG wave signal 621, the merged wave signal 63, the combined spectrum 91, the skin-color-pixel variation spectrum 92 (optional), and final spectrum 93 (optional).

If it is determined in step S105 that the difference between the average frequency and the strongest frequency does not exceed 5 BPM, it indicates that the current signal is stable. In the meantime, the processor 11 computes a weighted average of the strongest frequency and one or more frequencies whose frequency strength is greater than 50% of the strongest frequency and converts the weighted average into the heart rate in BPM, which is used as the heart rate output (step S106).

The above description is one of the spectrum analysis methods used in the heart-rate detecting method of the disclosure, and it is not limited to the method described above.

The heart-rate detecting method of the disclosure simultaneously uses at least two modified algorithms to generate the specific rPPG signals and performs the reverse merge process on the two rPPG signals. By the processes, a more obvious spectrum may be obtained after the FFT processing, so the accuracy of the heart rate detection results is enhanced.

What is claimed is:

1. A heart-rate detecting method implemented by a remote Photoplethysmography (rPPG), performed by an electronic device comprising at least one image-capturing unit and a processor, comprising:
step a) continuously capturing input frames based on continuous time series by the image-capturing unit, wherein the input frames comprise a facial image;
step b) computing, by the processor, multiple feature points of the facial image to obtain a skin-color average and storing the skin-color average and a timestamp corresponding to the skin-color average to a first queue;
step c) performing, by the processor, a Plane-Orthogonal-to-Skin (POS) algorithm and a Robust Pulse Rate from Chrominance-based rPPG (CHROM) algorithm based on the skin-color average of the multiple input frames in the first queue to respectively generate a first rPPG wave signal and a second rPPG wave signal;
step d) performing, by the processor, a reverse merge process on the first rPPG wave signal and the second rPPG wave signal to generate a merged wave signal;
step e) performing, by the processor, a Fast Fourier Transform (FFT) on the merged wave signal to generate a combined spectrum; and
step f) performing, by the processor, a spectrum analysis process on the combined spectrum to retrieve a heart rate output.

2. The heart-rate detecting method implemented by the rPPG of claim 1, after the step b) the method comprising:
step b11) performing a brightness detection process on the skin-color average and generating a brightness detection result; and
step b12) sending the brightness detection result to the image-capturing unit to adjust an exposure used by the image-capturing unit capturing a next input frame.

3. The heart-rate detecting method implemented by the rPPG of claim 1, wherein the first queue has a queue length M, and the first queue has a superimposition window, and a superimposition length N of the superimposition window is smaller than the queue length M, wherein after the step b) the method comprises:
step b21) when a frame quantity of the first queue is greater than the superimposition length N, performing the step c).

4. The heart-rate detecting method implemented by the rPPG of claim 1, wherein the step b) comprises:
step b01) identifying the multiple feature points in the facial image;
step b02) retrieving a skin-color value of the multiple feature points and multiple sampling points around the multiple feature points and computing an average of the skin-color value to obtain a skin-color base;
step b03) refining multiple skin-color pixels falling within a certain range of standard deviation of the skin-color base in a facial skin area, wherein the facial skin area is an image removing an eye part from the facial image; and
step b04) creating the skin-color averages according to the multiple skin-color pixels and storing the skin-color averages and the timestamps corresponding to the skin-color averages to the first queue.

5. The heart-rate detecting method implemented by the rPPG of claim 4, wherein the step b03) comprises refining the multiple skin-color pixels with the skin-color base are within positive or negative 1.5 times to positive or negative 3 times of the standard deviation.

6. The heart-rate detecting method implemented by the rPPG of claim 1, wherein the first queue has a queue length M, and the first queue has a superimposition window, wherein a superimposition length N of the superimposition window is smaller than the queue length M and the superimposition window is used to store latest N skin-color averages of the first queue, when the POS algorithm and the CHROM algorithm generate the first rPPG wave signal and a second rPPG wave signal, respectively superimposing the N skin-color averages in the superimposition window after being updated in the step b) on the latest N skin-color averages in the first queue.

7. The heart-rate detecting method implemented by the rPPG of claim 6, wherein before the POS algorithm and the CHROM algorithm output the first rPPG wave signal and a second rPPG wave signal, calibrating latest multiple frames of a first length D of the superimposition window, wherein a calibration level of each frame in each round is 1/D of an average difference of the multiple frames, and the first length D is smaller than the superimposition length N.

8. The heart-rate detecting method implemented by the rPPG of claim 7, wherein before the POS algorithm and the CHROM algorithm output the first rPPG wave signal and a second rPPG wave signal, performing an amplitude limiting process on the first rPPG wave signal and the second rPPG wave signal to make a positive or negative signal strength of the first rPPG wave signal and the second rPPG wave signal to be limited to one-half of an original signal strength to filter out a high-frequency noise and a low-frequency noise.

9. The heart-rate detecting method implemented by the rPPG of claim 1, wherein the reverse merge process takes a strongest strength from each frame node of the first rPPG wave signal and the second rPPG wave signal and takes a larger one of two absolute values of the two strongest strengths being taken as a signal strength of each frame node of the merged wave signal.

10. The heart-rate detecting method implemented by the rPPG of claim 1, comprising:
step g) after the step b), recording, by the processor, a total quantity of the skin-color pixels, computing a skin-color-pixel variation of a previous frame and a next frame, and storing the skin-color-pixel variation to a second queue;
step h) performing the FFT on the skin-color-pixel variation of the multiple input frames in the second queue to generate a skin-color-pixel variation spectrum; and
step i) after the step e), subtracting the skin-color-pixel variation spectrum from the combined spectrum to generate a final spectrum;
wherein the step f) is performed by the processor to perform the spectrum analysis process on the final spectrum to retrieve the heart rate output.

11. The heart-rate detecting method implemented by the rPPG of claim 1, wherein the step e) comprises performing, by the processor, the FFT on the merged wave signal to generate frequency information and normalizing an absolute value of the frequency information to obtain the combined spectrum, wherein the signal strength of multiple frequencies of the combined spectrum is between 0 and 1.

12. The heart-rate detecting method implemented by the rPPG of claim 11, wherein the spectrum analysis process comprises computing a real frequency represented by the combined spectrum according to the frame quantity and the timestamps stored in the first queue and retrieving the heart rate output according to a signal strength distribution of each frequency after retrieving the spectrum in a human heart rate range.

13. The heart-rate detecting method implemented by the rPPG of claim 11, wherein the step f) comprises:
step f1) not computing the heart rate output when there is no frequency whose frequency strength is greater than 0.5 in the combined spectrum.

14. The heart-rate detecting method implemented by the rPPG of claim 13, wherein the step f) comprises:
step f2) obtaining a strongest frequency of the combined spectrum;
step f3) obtaining one or more frequencies having a frequency strength greater than 50% of the strongest frequency in the combined spectrum; and
step f4) computing a weighted average of the strongest frequency and one or more frequencies whose frequency strength is greater than 50% of the strongest frequency, converting the weighted average into a heart rate in BPM, and taking the heart rate in BPM as the heart rate output.

15. The heart-rate detecting method implemented by the rPPG of claim 14, wherein the step f) comprises:
step f5) computing an average frequency of the combined spectrum; and
step f6) not computing the heart rate output when the difference between the average frequency and the strongest frequency exceeds 5 BPM.
